(19) United States
(12) Patent Application Publication
(10) Pub. No.: US 2011/0189734 A1
(43) Pub. Date: Aug. 4, 2011

(54) NOVEL METHODS AND CELL LINES

(76) Inventors: Alexander H. Taylor, King Of Prussia, PA (US); Yuan Zhu, King of Prussia, PA (US)

(21) Appl. No.: 12/673,526

(22) PCT Filed: Aug. 14, 2008
(86) PCT No.: PCT/US08/73114

§ 371 Date: Apr. 6, 2011

Related U.S. Application Data
(60) Provisional application No. 60/955,673, filed on Aug. 14, 2007.

(51) Int. Cl. C12P 21/00; C12N 15/87; C12N 5/10

(52) U.S. Cl. 435/69.6; 435/455; 435/326

(57) ABSTRACT

This invention relates to fermentation processes for producing antibodies and other antigen binding compounds. Specifically, the present invention provides methods for increasing the specific productivity of an antibody produced in cell culture, increasing cell growth and increasing viability of cells in cell culture. Also provided are, expression stable, viable cell lines comprising DNA encoding adenovirus GAM1 and capable of producing a monoclonal antibody or fragment and/or variant thereof.

### FIELD OF THE INVENTION

This invention relates to fermentation processes for producing antibodies and other biotherapeutic compounds.

### BACKGROUND OF THE INVENTION

Heterologous proteins are expressed in a variety of cell expression systems including bacterial, yeast and mammalian expression systems. For instance, monoclonal antibodies (IgG isotypes) are produced using a variety of expression systems including, host systems such as Chinese hamster ovary (CHO), NS0, hybridoma and myeloma cells or their derivatives. The amount of heterologous protein of interest produced in cell culture can often depend on co-expression of other proteins in the cell culture. The GAM1 protein of the avian CELO adenovirus activates transcription through inhibition of histone deacetylase 1 (HDAC1). Transient expression of GAM1 has been shown to increase reporter protein levels up to 4-fold in suspension cultures of CHO DG44 cells and up to 20-fold in recombinant CHO DG44-derived cell lines. Hacker, et al. Journal of Biotechnology 117 (2005) 21-29. However, Hacker, et al. also indicate that although GAM1 may have dramatic effects on heterologous protein expression in CHO cells it had no effect on stable IgG expression in sequentially transfected CHO cells.

Monoclonal antibodies such as those of the IgG type are currently used in therapy for a variety of diseases. In order for monoclonal antibodies to be produced for therapeutic purposes they must be made in sufficient quantities with purity and activity that meets regulatory requirements. Thus, there is a need to increase specific productivity of a monoclonal antibody in cell culture. The present invention discloses methods for increasing the specific productivity of an antibody produced in cell culture. Furthermore, the present invention provides methods for increasing cell growth and viability of cells in cell culture. Also provided are, expression stable, viable cell lines comprising DNA encoding GAM1 and capable of producing a monoclonal antibody or fragment and/or variant thereof.

### BRIEF DESCRIPTION OF THE DRAWINGS

FIG. 1: GAM1 Increased mAb Specific Production Rate (SPR) of Anti-OSM Cell Lines.

FIG. 2: The mRNA Level of GAM1 in Anti-OSM GAM1 cells.

FIG. 3: GAM1 Increased mAb SPR of Anti-MAG Cell Lines.

FIG. 4: GAM1 Increased mAb SPR of Anti-BAM Cell Lines.

FIG. 5: Anti-OSM GAM1 Clones Maintain High Productivity over 60 Passages.

FIG. 6: Anti-OSM Productivity Study—Viability of anti-OSM with GAM1.

FIG. 7: Anti-OSM Productivity Study—Growth of anti-OSM with GAM1.

FIG. 8: Anti-OSM Productivity Study—Titer of anti-OSM with GAM1.

FIGS. 9A and 9B: GAM1 Increased Cell Number and Viability which Results in the Increase of Mab Production in MR Culture.

FIG. 10: Anti-BAM Screening on 96-well BAM Clones.

FIG. 11: Anti-BAM Screening on 96-well BAM GAM1 clones.

### SUMMARY OF THE INVENTION

In one aspect of the present invention, methods are provided for increasing a first specific productivity of a recombinant antibody or fragment thereof in a first cell comprising co-expressing said antibody or fragment thereof with adenovirus GAM1 or a fragment and/or variant thereof wherein said first specific productivity is greater for said antibody or fragment thereof compared with a second specific productivity of the same antibody or fragment thereof expressed in a second cell in which said adenovirus GAM1 or fragment and/or variant thereof is not expressed.

In another aspect of the invention, methods are provided for increasing integral viable cells of a first cell culture comprising co-expressing an antibody or fragment thereof with adenovirus GAM1 or a fragment and/or variant thereof in at least one cell of said first cell culture wherein said integral viable cells of said first cell culture is greater compared with the integral viable cells of a second cell culture in which said adenovirus GAM1 or fragment and/or variant thereof is not expressed.

In yet another embodiment of the present invention, methods are provided for creating an expression stable, viable cell line comprising transfecting at least one mammalian cell with DNA encoding adenovirus GAM1 or a variant and/or fragment thereof wherein said expression stable, viable cell line comprises said at least one mammalian cell. In one aspect, the cell line remains viable and expression stable over at least about 40 passages, at least about 50 passages, or at least about 60 passages.

In yet another embodiment, expression stable, viable cell lines comprising DNA encoding adenovirus GAM1 or a fragment and/or a variant thereof and capable of producing a monoclonal antibody or fragment and/or variant thereof are provided.

### DETAILED DESCRIPTION OF THE INVENTION

### Glossary

A recombination site “native” to the genome, as used herein, means a recombination site that occurs naturally in the genome of a cell (i.e., the sites are not introduced into the genome, for example, by recombinant means.)

As used herein “enhancer/promoter” means region(s) of DNA (1) that drive initiation of transcription by providing a binding site for RNA polymerase and associated specific essential transcription factors (the promoter), and/or (2) comprise regulatory DNA elements that interact with genes to enhance their expression (enhancer). Example promoters include, but are not limited to, the Cytomegalovirus (CMV) immediate early promoter/enhancer, beta globin promoter, Rous sarcoma virus (RSV) long terminal repeat promoter/enhancer, and the elongation factor alpha promoter/enhancer.

As used herein “mammalian cell” may include but is not limited to any mammalian cell capable of continuous anchorage dependent or suspension growth, and capable of supporting recombinant protein expression. Examples of mammalian cells, include, but are not limited to, Chinese hamster ovary (CHO), PerC6, Her96, SP2/0, NS0, HeLa, Madin-Darby Canine Kidney, COS cells, baby hamster kidney cells, and NIH 3T3 cells.

As used herein “reporter nucleic acid construct” means a nucleic acid sequence that produces a selectable marker. Selectable markers include, but are not limited to, dihydrofolate reductase (dhfr), antibiotic resistance genes including, neomycin (neo), genticin, hygromycin B, puromycin, zeocin, and ampicillin, beta.-galactosidase, fluorescent protein, secreted form of human placental alkaline phosphatase, beta-glucuronidase, yeast selectable markers leu 2-d and URA3, apoptosis resistant genes, and antisense oligonucleotides.

“Host cell” is a cell, including but not limited to a mammalian cell, insect cell, bacterial cell or cell of a microorganism, that has been introduced (e.g., transformed, infected or transfected) or is capable of introduction (e.g., transformation, infection or transfection) by an isolated and/or heterologous polynucleotide sequence.

“Transformed” or “transforming” as known in the art, is a modification of an organism's genome or episome via the introduction of isolated and/or heterologous DNA, RNA, or DNA-RNA hybrid, or to any other stable introduction of such DNA or RNA.

“Transfected” or “transfecting” as known in the art, is the introduction of isolated and/or heterologous DNA, RNA, or a DNA-RNA hybrid, into a host cell or microorganism, including but not limited to recombinant DNA or RNA

“Heterologous(ly)” means (a) obtained from an organism through isolation and introduced into another organism, as, for example, via genetic manipulation or polynucleotide transfer, and/or (b) obtained from an organism through means other than those that exist in nature, and introduced into another organism, as for example, through cell fusion, induced mating, or transgenic manipulation. A heterologous material may, for example, be obtained from the same species or type, or a different species or type than that of the organism or cell into which it is introduced.

“Recombinant expression system(s)” refers to expression systems or portions thereof or polynucleotides of the invention introduced (e.g, transfected, infected, or transformed) into a host cell or host cell lysate for the production of the polynucleotides and polypeptides of the invention.

As used herein, an “artificial chromosome” is a nucleic acid molecule that can stably replicate and segregate alongside endogenous chromosomes in a cell. It has the capacity to act as a gene delivery vehicle by accommodating and expressing foreign genes contained therein. A mammalian artificial chromosome (MAC) refers to chromosomes that have an active mammalian centromere(s). Plant artificial chromosomes, insect artificial chromosomes, yeast artificial chromosomes and avian artificial chromosomes refer to chromosomes that include plant, insect, yeast and avian centromeres, respectively. A human artificial chromosome (HAC) refers to chromosomes that include human centromeres. For exemplary artificial chromosomes, see, e.g., U.S. Pat. Nos. 6,025,155; 6,077,697; 5,288,625; 5,712,134; 5,695,967; 5,869,294; 5,891,691 and 5,721,118 and published International PCT application Nos, WO 97/40183 and WO 98/08964.

As used herein, the term “satellite DNA-based artificial chromosome (SATAC)” is interchangable with the term “artificial chromosome expression system (ACes).” These artificial chromosomes are substantially all neutral non-coding sequences (heterochromatin) except for foreign heterologous, typically gene-encoding nucleic acid, that is interspersed within the heterochromatin for the expression therein (see U.S. Pat. Nos. 6,025,155 and 6,077,697 and International PCT application No. WO 97/40183). Foreign genes contained in these artificial chromosome expression systems can include, but are not limited to, nucleic acid that encodes traceable marker proteins (reporter genes), such as fluorescent proteins, such as green, blue or red fluorescent proteins (GFP, BFP and RFP, respectively), other reporter genes, such as beta-galactosidase and proteins that confer drug resistance, such as a gene encoding hygromycin-resistance. Other examples of heterologous DNA include, but are not limited to, DNA that encodes therapeutically effective substances, such as anti-cancer agents, enzymes and hormones, and DNA that encodes other types of proteins, such as antibodies.

As used herein “recombinant antibody” means all variants of an antibody and/or fragment thereof expressed in a host cell from a recombinant expression system, including all modified and unmodified antibodies and/or fragment and/or variants thereof. Chemical modifications of a recombinant antibody may include, but are not limited to, methionine oxidation, glycosylation, gluconoylation, N-terminal glutamine cyclization and deamidation, and asparagine deamidation.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term “wild type” as is understood in the art refers to a polypeptide or polynucleotide sequence that occurs in a native population without mutation. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which typically include different antibodies directed against different epitopes, each monoclonal antibody is directed against a single epitope on the antigen.

“Antibody fragments” comprise a portion of a full length antibody, generally the antigen binding or variable domain thereof. Examples of antibody fragments include Fab, Fab′, F(ab′)₂, and Fv fragments; diabodies; linear antibodies; single-chain antibody molecules, and multispecific antibodies formed from antibody fragments.

The term “antigen binding fragment” refers to the domain of an antibody that specifically binds to an antigen.

The phrase “immunoglobulin single variable domain” refers to an antibody variable region (e.g., V_{H}, V_{HH}, V_{L}) that specifically binds an antigen or epitope independently of other V regions or domains; however, as the term is used herein, an immunoglobulin single variable domain can be present in a format (e.g., homo- or hetero-multimer) with other variable regions or variable domains where the other regions or domains are not required for antigen binding by the single immunoglobulin variable domain (i.e., where the immunoglobulin single variable domain binds antigen independently of the additional variable domains). “Immunoglobulin single variable domain” encompasses not only an isolated antibody single variable domain polypeptide, but also larger polypeptides that comprise one or more monomers of an antibody single variable domain polypeptide sequence. A “domain antibody” or “dAb” is the same as an “immunoglobulin single variable domain” polypeptide as the term is used herein. An immunoglobulin single variable domain polypeptide, as used herein refers to a mammalian immunoglobulin single variable domain polypeptide, which may be human, but also includes rodent (for example, as disclosed in WO 00/29004, the contents of which are incorporated herein by reference in their entirety) or camelid V_{HH} dAbs. Camelid dAbs are immunoglobulin single variable domain polypeptides which are derived from species including camel, llama, alpaca, dromedary, and guanaco, and comprise heavy chain antibodies naturally devoid of light chain: V_{HH}. V_{HH} molecules are about ten times smaller than IgG molecules, and as single polypeptides, they are very stable, resisting extreme pH and temperature conditions.

As used herein, “titer yield” refers to the concentration of a product (e.g., heterologously expressed polypeptide and/or recombinant antibody) in solution (e.g., culture broth or cell-lysis mixture or buffer) and may be expressed as mg/L or g/L. An increase in titer yield may refer to an absolute or relative increase in the concentration of a product produced under two defined set of conditions.

As used herein “integral viable cells” refers to the area found under a growth curve comprised of a vertical axis representing the number of viable cells, and a horizontal axis representing number of culture days, and can be expressed in units of cell-days.

As used herein “specific productivity” refers to the productivity of a cell line producing a heterologous protein, for instance an IgG, in cell culture. Specific productivity can be calculated by dividing the final yield by the integral viable cells, and is expressed in units of pg/cell/day.

As used herein “simultaneous co-transfection” or “simultaneously co-transfected” refers to methods of co-transfecting cells or cells that are co-transfected at the same time with DNA encoding at least one first heterologous protein (e.g., an antibody or fragment thereof, including a heavy chain and/or a light chain of an antibody) and with DNA encoding at least one second heterologous protein (e.g., GAM1) and culturing in the presence of selectable medium. By way of example, DNA encoding the heavy chain of an antibody may be simultaneously transfected into a cell with DNA encoding GAM1. As another example, DNA encoding heavy and DNA encoding light chain of an antibody may be simultaneously transected into a cell with DNA encoding GAM1. As used herein “simultaneous co-transfection expression” refers the expression of at least one first heterologous protein (e.g., an antibody or fragment thereof, including a heavy chain and/or a light chain of an antibody) in a cell that has been simultaneously co-transfected.

As used herein “sequential transfection” or “sequentially transfected” refers to methods of transfecting cells or cells transfected with DNA encoding at least one first heterologous protein (e.g., an antibody or fragment thereof, including a heavy chain and/or a light chain of an antibody) and then subsequently transfecting cells with DNA encoding at least one second heterologous protein (e.g., GAM1) and culturing in the presence of selectable medium. By way of example, DNA encoding the heavy and/or light chain of an antibody is transfected into the genome of a cell creating a stable viable cell line that expresses the heavy and/or light chain. Cells that are transfected can be selected using a selection marker. DNA encoding GAM1 is then transfected into the stable viable cell line, which can be selected using a selectable medium. By way of another example, DNA encoding GAM1 may be transfected into the genome of a cell creating a stable viable cell line that expresses GAM1. DNA encoding the heavy and/or light chain of an antibody is then transfected into the stable viable GAM1 cell line. Cells that are transfected can be selected using a selection marker. As used herein “sequential transfection expression” refers to the expression of at least one first heterologous protein in a cell that has been sequentially transfected.

As used herein “stably integrated” or “stable integration” means a transformed (or transformation of) a heterologous nucleic acid of interest into a host cell's chromosomal DNA such that long-term, reproducible expression is achieved.

As used herein “stable expression” or “expression stable” refers to expression from any cell that is capable of sustaining expression of at least one recombinant protein. Expression stable cells include, but are not limited to, simultaneously co-transfected cells and sequentially transfected cells.

As used herein “passage” or “passaged” refers to an interval of time necessary for cells in culture to produce a new generation of cells and still remain viable. By way of example, CHO cells described in the present invention may undergo one passage every 3 to 4 days. Therefore, cells which undergo one passage every 3 to 4 days and which undergo 50 passages remain viable for at least 175 days.

“Isolated” means altered “by the hand of man” from its natural state, has been changed or removed from its original environment, or both. For example, a polynucleotide or a polypeptide naturally present in a living organism is not “isolated,” but the same polynucleotide or polypeptide separated from the coexisting materials of its natural state is “isolated”, including but not limited to when such polynucleotide or polypeptide is introduced back into a cell, even if the cell is of the same species or type as that from which the polynucleotide or polypeptide was separated.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Polynucleotide(s)” generally refers to any polyribonucleotide or polydeoxyribonucleotide that may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that comprise one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may comprise amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may comprise many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, GPI anchor formation, hydroxylation, iodination, methylation, myristoylation, oxidation, proteolytic processing, phosphorylation, prenylation, raccmization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, PROTEINS—STRUCTURE AND MOLECULAR PROPERTIES, 2nd Ed., T. E. Creighton, W. H. Freeman and Company, New York (1993) and Wold, F., Posttranslational Protein Modifications Perspectives and Prospects, pgs. 1-12 in POSTTRANSLATIONAL COVALENT MODIFICATION OF PROTEINS, B. C. Johnson, Ed., Academic Press, New York (1983); Seifter et al., Meth. Enzymol. 182:626-646 (1990) and Rattan et al., Protein Synthesis: Posttranslational Modcations and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

“Identity,” means, for polynucleotides and polypeptides, as the case may be, the comparison calculated using an algorithm provided in (1) and (2) below.

(1) Identity for polynucleotides is calculated by multiplying the total number of nucleotides in a given sequence by the integer defining the percent identity divided by 100 and then subtracting that product from said total number of nucleotides in said sequence, or:

nₙ≦xₙ−(xₙ·y),

wherein nₙ is the number of nucleotide alterations, xₙ is the total number of nucleotides in a given sequence, y is 0.95 for 95%, 0.97 for 97% or 1.00 for 100%, and · is the symbol for the multiplication operator, and wherein any non-integer product of xₙ and y is rounded down to the nearest integer prior to subtracting it from xₙ. Alterations of a polynucleotide sequence encoding a polypeptide may create nonsense, missense or frameshift mutations in this coding sequence and thereby alter the polypeptide encoded by the polynucleotide following such alterations.

(2) Identity for polypeptides is calculated by multiplying the total number of amino acids by the integer defining the percent identity divided by 100 and then subtracting that product from said total number of amino acids, or:

nₐ≦xₐ−(xₐ·y),

wherein nₐ is the number of amino acid alterations, xₐ is the total number of amino acids in the sequence, y is 0.95 for 95%, 0.97 for 97% or 1.00 for 100%, and · is the symbol for the multiplication operator, and wherein any non-integer product of xₐ and y is rounded down to the nearest integer prior to subtracting it from xₐ.

“Variant(s)” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusion proteins and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. The present invention also includes include variants of each of the polypeptides of the invention, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Several, 5-10, 1-5, 1-3, 1-2 or 1 amino acids may be substituted, deleted, or added in any combination. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

Intact antibodies include heteromultimeric proteins and glycoproteins comprising at least two heavy and two light chains. Aside from IgM, intact antibodies are usually heterotetrameric glycoproteins of approximately 150 Kda, composed of two identical light (L) chains and two identical heavy (H) chains. Typically, each light chain is linked to a heavy chain by one covalent disulfide bond while the number of disulfide linkages between the heavy chains of different immunoglobulin isotypes varies. Each heavy and light chain also has intrachain disulfide bridges. Each heavy chain has at one end a variable domain (VH) followed by a number of constant regions. Each light chain has a variable domain (VL) and a constant region at its other end; the constant region of the light chain is aligned with the first constant region of the heavy chain and the light chain variable domain is aligned with the variable domain of the heavy chain. The light chains of antibodies from most vertebrate species can be assigned to one of two types called Kappa and Lambda based on the amino acid sequence of the constant region. Depending on the amino acid sequence of the constant region of their heavy chains, human antibodies can be assigned to five different classes, IgA, IgD, IgE, IgG and IgM. IgG and IgA can be further subdivided into subclasses, IgG1, IgG2, IgG3 and IgG4; and IgA1 and IgA2. Species variants exist with mouse and rat having at least IgG2a, IgG2b. The variable domain of the antibody confers binding specificity upon the antibody with certain regions displaying particular variability called complementarity determining regions (CDRs). The more conserved portions of the variable region are called Framework regions (FR). The variable domains of intact heavy and light chains each comprise four FR connected by three CDRs. The CDRs in each chain are held together in close proximity by the FR regions and with the CDRs from the other chain contribute to the formation of the antigen binding site of antibodies. The constant regions are not required to be directly involved in the binding of the antibody to the antigen but exhibit various effector functions such as participation in antibody dependent cell-mediated cytotoxicity (ADCC), phagocytosis via binding to Fey receptor, half-life/clearance rate via neonatal Fc receptor (FcRn) and complement dependent cytotoxicity via the C1q component of the complement cascade.

Human antibodies may be produced by a number of methods known to those of skill in the art. Human antibodies can be made by the hybridoma method using human myeloma or mouse-human heteromyeloma cells lines see Kozbor J. Immunol. 133, 3001, (1984) and Brodeur, Monoclonal Antibody Production Techniques and Applications, pp 51-63 (Marcel Dekker Inc, 1987). Alternative methods include the use of phage libraries or transgenic mice both of which utilize human V region repertories (see Winter G, (1994), Annu. Rev. Immunol. 12, 433-455, Green L L (1999), J. Immunol. Methods 231, 11-23).

Several strains of transgenic mice are now available wherein their mouse immunoglobulin loci has been replaced with human immunoglobulin gene segments (see Tomizuka K, (2000) PNAS 97:722-727; Fishwild D. M (1996) Nature Biotechnol. 14, 845-851, Mendez M J, 1997, Nature Genetics, 15, 146-156). Upon antigen challenge such mice are capable of producing a repertoire of human antibodies from which antibodies of interest can be selected. Of particular note is the Trimera™ system (see Eren R et al, (1998) Immunology 93:154-161) where human lymphocytes are transplanted into irradiated mice, the Selected Lymphocyte Antibody System (SLAM, see Babcook et al, PNAS (1996) 93:7843-7848) where human (or other species) lymphocytes are effectively put through a massive pooled in vitro antibody generation procedure followed by deconvulated, limiting dilution and selection procedure and the Xenomouse II™ (Abgenix Inc). An alternative approach is available from Morphotek Inc using the Morphodoma™ technology.

Phage display technology can be used to produce human antibodies (and fragments thereof), see McCafferty; Nature, 348, 552-553 (1990) and Griffiths A D et at (1994) EMBO 13:3245-3260. According to this technique antibody V domain genes are cloned in frame into either a major or minor coat of protein gene of a filamentous bacteriophage such as M13 or fd and displayed (may be with the aid of a helper phage) as functional antibody fragments on the surface of the phage particle. Selections based on the functional properties of the antibody result in selection of the gene encoding the antibody exhibiting those properties. The phage display technique can be used to select antigen specific antibodies from libraries made from human B cells taken from individuals afflicted with a disease or disorder described above or alternatively from unimmunized human donors (see Marks; J. Mol. Bio. 222:581-597, 1991). Where an intact human antibody is desired comprising a Fc domain it is necessary to reclone the phage displayed derived fragment into a mammalian expression vectors comprising the desired constant regions and establishing stable expressing cell lines.

The technique of affinity maturation (Marks, Bio/technol 10:779-783 (1992)) may be used to improve binding affinity wherein the affinity of the primary human antibody is improved by sequentially replacing the H and L chain V regions with naturally occurring variants and selecting on the basis of improved binding affinities. Variants of this technique such as “epitope imprinting” are now also available see WO 93/06213. See also Waterhouse; Nucl. Acids Res. 21:2265-2266 (1993).

The use of intact non-human antibodies in the treatment of human diseases or disorders carries with it the potential for the now well established problems of immunogenicity; that is the immune system of the patient mounts a neutralizing response against the non-human antibody. Immunogenicity is particularly evident upon multiple administration of the non-human antibody to a human patient. Various techniques have been developed over the years to overcome these problems. One such technique includes chimeric antibodies, which generally comprise a non-human (e.g. rodent such as mouse) variable domain fused to a human constant region. Because the antigen-binding site of an antibody is localized within the variable regions the chimeric antibody retains its binding affinity for the antigen but acquires the effector functions of the human constant region and are therefore able to perform effector functions such as described supra. Chimeric antibodies are typically produced using recombinant DNA methods. DNA encoding the antibodies (e.g. cDNA) is isolated and sequenced using conventional procedures (e.g. by using oligonucleotide probes that are capable of binding specifically to genes encoding the H and L chains of the antibody of the invention). Hybridoma cells serve as a typical source of such DNA. Once isolated, the DNA is placed into expression vectors which are then transfected into host cells such as E. coli, COS cells, CHO cells or myeloma cells that do not otherwise produce immunoglobulin protein to obtain synthesis of the antibody. The DNA may be modified by substituting the coding sequence for human L and H chains for the corresponding non-human (e.g. murine) H and L constant regions see e.g. Morrison, PNAS 81:6851 (1984).

Antibodies of the present invention maybe produced as a fusion protein with a heterologous signal sequence having a specific cleavage site at the N terminus of the mature protein. The signal sequence should be recognized and processed by the host cell. For prokaryotic host cells, the signal sequence may be an alkaline phosphatase, penicillinase, or heat stable enterotoxin 11 leaders. For yeast secretion the signal sequences may be a yeast invertase leader, a factor leader or acid phosphatase leaders see e.g. WO90/13646. In mammalian cell systems, viral secretory leaders such as herpes simplex gD signal and a native immunoglobulin signal sequence are available. A signal sequence may be ligated in reading frame to DNA encoding the antibody of the invention.

Origin of replications are well known in the art with pBR322 suitable for most gram-negative bacteria, 2μ plasmid for most yeast and various viral origins such as SV40, polyoma, adenovirus, VSV or BPV for most mammalian cells. An origin of replication component may not be not needed for mammalian expression vectors but the SV40 may be used since it contains the early promoter.

Selection genes encode proteins that (a) confer resistance to antibiotics or other toxins e.g. ampicillin, neomycin, methotrexate or tetracycline or (b) complement auxiotrophic deficiencies or supply nutrients not available in the complex media. A selection scheme may involve arresting growth of the host cell. Cells, which have been successfully transformed with the genes encoding the therapeutic antibody of the present invention, survive due to e.g. drug resistance conferred by the selection marker. Another example is the so-called DHFR selection marker wherein transformants are cultured in the presence of methotrexate. In typical embodiments, cells are cultured in the presence of increasing amounts of methotrexate to amplify the copy number of the exogenous gene of interest. CHO cells are a particularly useful cell line for the DHFR selection. A further example is the glutamate synthetase expression system (Lonza Biologics). A suitable selection gene for use in yeast is the trp1 gene, see Stinchcomb, et al., Nature 282:38, 1979.

Suitable promoters for expressing antibodies are operably linked to DNA/polynucleotide encoding the antibody. Promoters for prokaryotic hosts include, but are not limited to, phoA promoter, Beta-lactamase and lactose promoter systems, alkaline phosphatase, tryptophan and hybrid promoters such as Tac. Promoters suitable for expression in yeast cells include, but are not limited to, 3-phosphoglycerate kinase or other glycolytic enzymes e.g. enolase, glyceralderhyde 3 phosphate dehydrogenase, hexokinase, pyruvate decarboxylase, phosphofructokinase, glucose 6 phosphate isomerase, 3-phosphoglycerate mutase and glucokinase. Inducible yeast promoters include, but are not limited to, alcohol dehydrogenase 2, isocytochrome C, acid phosphatase, metallothionein and enzymes responsible for nitrogen metabolism or maltose/galactose utilization.

Promoters for expression in mammalian cell systems include, but are not limited to, viral promoters such as polyoma, fowlpox and adenoviruses (e.g. adenovirus 2), bovine papilloma virus, avian sarcoma virus, cytomegalovirus (in particular the immediate early gene promoter, for example wherein the 5′ untranslated region of the HCMV IE gene comprising exon 1 is included and intron A is partially or completely excluded as described in WO 02/36792), retrovirus, hepatitis B virus, actin, rous sarcoma virus (RSV) promoter and the early or late Simian virus 40. Of course the choice of promoter is based upon suitable compatibility with the host cell used for expression. In one embodiment therefore there is provided a first plasmid comprising a RSV and/or SV40 and/or CMV promoter, DNA encoding light chain V region (V_{L}) of the invention, κC region together with neomycin and ampicillin resistance selection markers and a second plasmid comprising a RSV or SV40 promoter, DNA encoding the heavy chain V region (V_{H}) of the invention, DNA encoding the γ1 constant region, DHFR and ampicillin resistance markers. In another aspect, the promoter is not pEF (elongation factor alpha).

Certain embodiments for expression in higher eukaroytics comprise an enhancer element operably linked to the promoter element in a vector may be used. Suitable mammalian enhancer sequences include enhancer elements from globin, elastase, albumin, fetoprotein and insulin. Alternatively, one may use an enhancer element from a eukaroytic cell virus such as SV40 enhancer (at bp100-270), cytomegalovirus early promoter enhancer, polyma enhancer, baculoviral enhancer or murine IgG2a locus (see WO04/009823). The enhancer may be located on the vector at a site upstream to the promoter.

Suitable host cells for cloning or expressing vectors encoding antibodies are prokaroytic, yeast or higher eukaryotic cells. Suitable prokaryotic cells include eubacteria e.g. enterobacteriaceae such as Escherichia e.g. E. Coli (for example ATCC 31,446; 31,537; 27,325), Enterobacter, Erwinia, Klebsiella Proteus, Salmonella e.g. Salmonella typhimurium, Serratia e.g. Serratia marcescans and Shigella as well as Bacilli such as B. subtilis and B. lichenifonnis (see DD 266 710), Pseudomonas such as P. aeruginosa and Streptomyces. Of the yeast host cells, Saccharomyces cerevisiae, schizosaccharomyces pombe, Kluyveromyces (e.g. ATCC 16,045; 12,424; 24178; 56,500), yarrowia (EP402, 226), Pichia Pastoris (EP183, 070, see also Peng et at J. Biotechnol. 108 (2004) 185-192), Candida, Trichoderma reesia (EP244, 234), Penicillin, Tolypocladium and Aspergillus hosts such as A. nidulans and A. niger are also contemplated.

Suitable higher eukaryotic host cells include, but are not limited to, mammalian cells such as COS-1 (ATCC No. CRL 1650) COS-7 (ATCC CRL 1651), human embryonic kidney line 293, baby hamster kidney cells (BHK) (ATCC CRL. 1632), BHK570 (ATCC NO: CRL 10314), 293 (ATCC NO. CRL 1573), Chinese hamster ovary cells CHO (e.g. CHO-K1, ATCC NO: CCL 61, DHFR-CHO cell line such as DG44 (see Urlaub et al, (1986) Somatic Cell Mol. Genet. 12, 555-556)), particularly those CHO cell lines adapted for suspension culture, mouse sertoli cells, monkey kidney cells, African green monkey kidney cells (ATCC CRL-1587), HELA cells, canine kidney cells (ATCC CCL 34), human lung cells (ATCC CCL 75), Hep G2 and myeloma or lymphoma cells e.g. NS0 (see U.S. Pat. No. 5,807,715), Sp2/0, Y0.

“Microorganism(s)” means a (1) prokaryote, including but not limited to, (a) Bacteria(l)(um), meaning a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis, (b) an archacon, including but not limited to Archaebacter, and (2) a unicellular or filamentous eukaryote, including but not limited to, a protozoan, a fungus, a member of the genus Saccharomyces, Kluveromyces, or Candida, and a member of the species Saccharomyces ceriviseae, Kluveromyces lactis, or Candida albicans.

As used herein “harvesting” cells refers to collection of cells from cell culture. Cells may be concentrated during harvest to separate them from culture broth, for instance by centrifugation or filtration. Harvesting cells may further comprise the step of lysing the cells to obtain intracellular material, such as, but not limited to polypeptides and polynucleotides. It should be understood by the skilled artisan that certain cellular material, including but not limited to, heterologously expressed polypeptide, may by released from cells during culture. Thus, a product (e.g., a heterologously expressed polypeptide such as an antibody or fragment thereof) of interest may remain in culture broth after cells are harvested.

Host cells transformed with vectors encoding therapeutic antibodies or antigen binding fragments thereof may be cultured by any method known to those skilled in the art. Host cells may be cultured in spinner flasks, roller bottles or hollow fibre systems but it is preferred for large scale production that stirred tank reactors are used particularly for suspension cultures. The stirred tanks are adapted for aeration using e.g. spargers, baffles or low shear impellers. For bubble columns and airlift reactors direct aeration with air or oxygen bubbles maybe used. Where the host cells are cultured in a serum free culture media the media may be supplemented with a cell protective agent such as pluronic F-68 to help prevent cell damage as a result of the aeration process. Depending on the host cell characteristics, either microcarriers maybe used as growth substrates for anchorage dependent cell lines or the cells maybe adapted to suspension culture (which is typical). The culturing of host cells, particularly invertebrate host cells may utilise a variety of operational modes such as fed-batch, repeated batch processing (see Drapeau et al (1994) cytotechnology 15: 103-109), extended batch process or perfusion culture. Although recombinantly transformed mammalian host cells may be cultured in serum-containing media such as fetal calf serum (FCS), such host cells may be cultured in synthetic serum-free media such as disclosed in Keen et al (1995) Cytotechnology 17:153-163, or commercially available media such as ProCHO-CDM or UltraCHO™ (Cambrex N.J., USA), supplemented where necessary with an energy source such as glucose and synthetic growth factors such as recombinant insulin. The serum-free culturing of host cells may require that those cells are adapted to grow in serum free conditions. One adaptation approach is to culture such host cells in serum containing media and repeatedly exchange 80% of the culture medium for the serum-free media so that the host cells learn to adapt in serum free conditions (see e.g. Scharfenberg K et al (1995) in Animal Cell technology: Developments towards the 21st century (Beuvery E. C. et al eds), pp 619-623, Kluwer Academic publishers).

Antibodies secreted into the media may be recovered and purified using a variety of techniques to provide a degree of purification suitable for the intended use. For example, the use of therapeutic antibodies for the treatment of human patients typically mandates at least 95% purity, more typically 98% or 99% or greater purity (compared to the crude culture medium). In the first instance, cell debris from the culture media is typically removed using centrifugation followed by a clarification step of the supernatant using e.g. microfiltration, ultrafiltration and/or depth filtration. A variety of other techniques such as dialysis and gel electrophoresis and chromatographic techniques such as hydroxyapatite (HA), affinity chromatography (optionally involving an affinity tagging system such as polyhistidine) and/or hydrophobic interaction chromatography (HIC, see U.S. Pat. No. 5,429,746) are available. Antibodies may be purifies by following various clarification steps, are captured using Protein A or G affinity chromatography followed by further chromatography steps such as ion exchange and/or HA chromatography, anion or cation exchange, size exclusion chromatography and ammonium sulphate precipitation. Typically, various virus removal steps are also employed (e.g. nanofiltration using e.g. a DV-20 filter). Following these various steps, a purified preparation which may be monoclonal comprising at least 75 mg/ml or greater e.g. 100 mg/ml or greater of the antibody of the invention or antigen binding fragment thereof is provided and therefore forms an embodiment of the invention. Suitably such preparations are substantially free of aggregated forms of antibodies.

Thus, in one embodiment methods are provided for increasing a first specific productivity of a recombinant antibody or fragment thereof in a first cell comprising co-expressing said antibody or fragment thereof with adenovirus GAM1 or a fragment and/or variant thereof wherein said first specific productivity is greater for said antibody or fragment thereof compared with a second specific productivity of the same antibody or fragment thereof expressed in a second cell in which adenovirus GAM1 or fragment and/or variant thereof is not expressed. The antibodies of the present invention may be whole antibodies, monoclonal antibodies, humanized antibodies, fully human antibodies, antibody fragments, and/or domain antibodies. The first cell may be a mammalian cell. Mammalian cells include, but are not limited to, Chinese hamster ovary (CHO), PerC6, Her96, SP2/0, NS0, HeLa, Madin-Darby Canine Kidney, COS cells, baby hamster kidney cells, and NIH 3T3 cells. Said first cell may comprise an artificial chromosome and/or episome DNA. In one aspect, said first and second cell are the same type of host cell. The first and/or second cell may be in culture.

In another aspect of the present invention, said adenovirus GAM1 is avian. Said adenovirus GAM1 or fragment and/or variant thereof may be expressed from a gene transformed into the genome of said host cell and/or it may be expressed from a vector. Adenovirus GAM1 may be present in more than one copy. Adenovirus GAM1 may be a wild type or adenovirus GAM1 may be a variant or fragment of wild type adenovirus GAM1. In another aspect, a variant or fragment of wild type adenovirus GAM1 increases the specific productivity of said monoclonal antibody or fragment thereof more than wild type adenovirus GAM1. In yet another aspect, the first cell is stably transfected with DNA encoding said antibody or antibody fragment and/or DNA encoding said adenovirus GAM1 or fragment and/or variant thereof. In another embodiment, said adenovirus GAM1 or fragment and/or variant thereof is expressed from a vector in said first cell. Adenovirus GAM1 or fragment and/or variant thereof may be present in more than one copy. Adenovirus GAM1 may be a variant of wild type adenovirus GAM1. Some variant of wild type adenovirus GAM1 may increase the specific productivity of said monoclonal antibody or fragment thereof more than wild type adenovirus GAM1. Adenovirus GAM1 expression can be measured by various methods understood in the art. For instance, GAM1 expression can be measured by assessing GAM1 mRNA production in at least one cell. GAM1 expression can be high, intermediate or low. Messenger RNA levels have been used to determine production of corresponding proteins (G. Bevilacqua, M. E. Sobel, L. A. Liotta and T. S. Steeg, Cancer Res. 49, 5185-5190 (1989)).

The first cell may be sequentially transfected with DNA encoding said antibody or antibody fragment and DNA encoding said adenovirus GAM1 or a fragment and/or variant thereof. Alternatively, the first cell may be simultaneously co-transfected with DNA encoding said antibody or antibody fragment and DNA encoding said adenovirus GAM1 or a fragment and/or variant thereof.

In another aspect of the present invention, methods are provided for increasing integral viable cells of a first cell culture comprising co-expressing an antibody or fragment thereof with adenovirus GAM1 or a fragment and/or variant thereof in at least one cell of said first cell culture wherein said integral viable cells of said first cell culture is greater compared with integral viable cells of a second cell culture in which adenovirus GAM1 or fragment and/or variant thereof is not expressed. The antibody may be a whole antibody, a monoclonal antibody, a humanized antibody, a fully human antibody, an antibody fragment, and/or a domain antibody. The first cell culture may be a mammalian cell culture. Mammalian cell culture may include, but is not limited to, Chinese hamster ovary (CHO), PerC6, Her96, SP2/0, NS0, HeLa, Madin-Darby Canine Kidney, COS cells, baby hamster kidney cells, and NIH 3T3 cells. At least one cell in cell culture may comprise an artificial chromosome and/or episome DNA. In one aspect, said first and second cell culture comprise the same type of host cell.

In another aspect of the present invention, said adenovirus GAM1 is of canine, bovine, murine, ovine, porcine, avian or simian origin, in one aspect said adenovirus GAM1 is avian. Said adenovirus GAM1 or fragment and/or variant thereof may be expressed from a gene transformed into the genome of said host cell and/or it may be expressed from a vector. Adenovirus GAM1 or fragment and/or variant thereof may be present in more than one copy. Adenovirus GAM1 may be a wild type or adenovirus GAM1 may be a variant or fragment of wild type adenovirus GAM1. In another aspect a variant or fragment of wild type adenovirus GAM1 increases the integral viable cells more than wild type adenovirus GAM1. In yet another aspect, the recombinant protein producing cells are stably transfected with DNA encoding said recombinant antibody and DNA encoding GAM1. In another embodiment the recombinant protein producing cells are transiently transfected with DNA encoding said recombinant antibody and DNA encoding GAM1 or a fragment and/or variant thereof. Stably transfected cells can be recovered from the transient transfection by application of selective pressure, e.g., neomycin (G418).

In yet another embodiment of the present invention, methods are provided for creating an expression stable, viable cell line comprising transfecting at least one mammalian cell with DNA encoding GAM1 or a fragment and/or variant thereof wherein said stable expressing, viable cell line comprises said at least one mammalian cell. In one aspect the cell line remains viable over at least about 40 passages, at least about 50 passages, or at least about 60 passages. In another aspect, at least 1 passage occurs every 3 to 4 days. The cell line may remain viable for at least 210 days. At least one mammalian cell may be selected from the group of Chinese hamster ovary (CHO), PerC6, Her96, SP2/0, NS0, HeLa, Madin-Darby Canine Kidney, COS cells, baby hamster kidney cells, and NIH 3T3 cells. The mammalian cell may be simultaneously co-transfected or sequentially transfected with DNA encoding an antibody or fragment thereof and with DNA encoding GAM1 or a fragment and/or variant thereof.

In addition, methods are provided for increasing integral viable cells of a first cell culture comprising co-expressing an antibody or fragment thereof with adenovirus GAM1 or a fragment and/or variant thereof in at least one cell of said first cell culture wherein said integral viable cells of said first cell culture is greater compared with the integral viable cells of a second cell culture in which said adenovirus GAM1 or a fragment and/or variant thereof is not expressed, wherein the amount of integrally viable cells is increased because of cell density. In some embodiments, the cell density is increased because of the expression of adenovirus GAM1. In another embodiment, the amount of integrally viable cells is increased because of prolonged growth period.

Also provided are expression stable, viable cell lines comprising DNA encoding adenovirus GAM1 or a fragment and/or variant thereof and capable of producing a monoclonal antibody or fragment and/or variant thereof.

The following examples illustrate various aspects of this invention. These examples do not limit the scope of this invention which is defined by the appended claims

### EXAMPLES

### Example 1

### Increased Specific Productivity (SPR) of Monoclonal Antibodies by Transfection of GAM1 in mAb Expressing CHO Cells

A CHO cell line was previously made and produced anti-Oncostatin M (OSM) monoclonal antibody at an intermediate level (SPR=11 pg/cell/day). The heavy chain and light chain were both regulated by RSV_LTR promoters. To determine if GAM1 can increase mAb expression in CHO_DG44 cells, the cells were sequentially stably transfected with pcDNA_GAM1 which contained the wild-type GAM1 cDNA regulated by a CMV promoter and zeomycin selection gene. After 20 days selection in 96 well plates, the Zeo-resistant clones were grown up and sequentially transferred to 24 6-well plates, and then into T-25 flasks. To measure specific productivity, the cells were spun down and re-cultured in fresh medium. After 24 hours, the cells were counted and the supernatants were collected for ELISA analysis. The specific productivity was calculated as titer/cell number/1 day (pg/cell/day).

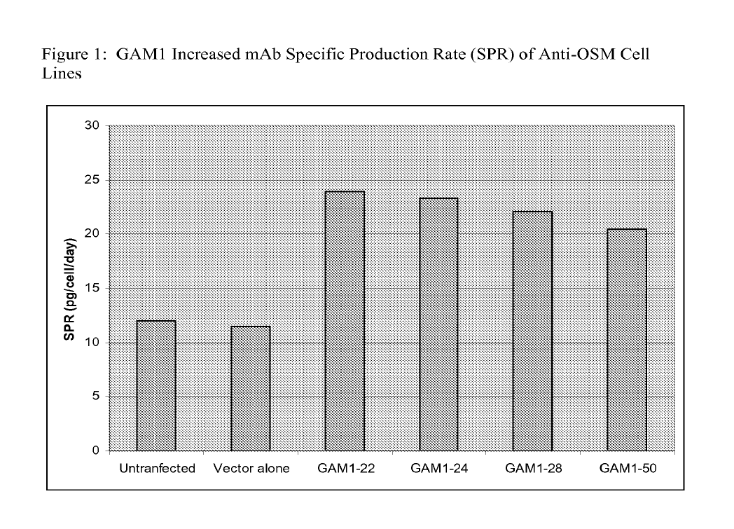

About 50-100% increase of mAb specific productivity was observed among the GAM1 transfected clones designated as GAM1-22, 24, 28 and 50 compared with the original line (untranfected) and vector alone transfected line (vector alone) as shown in FIG. 1.

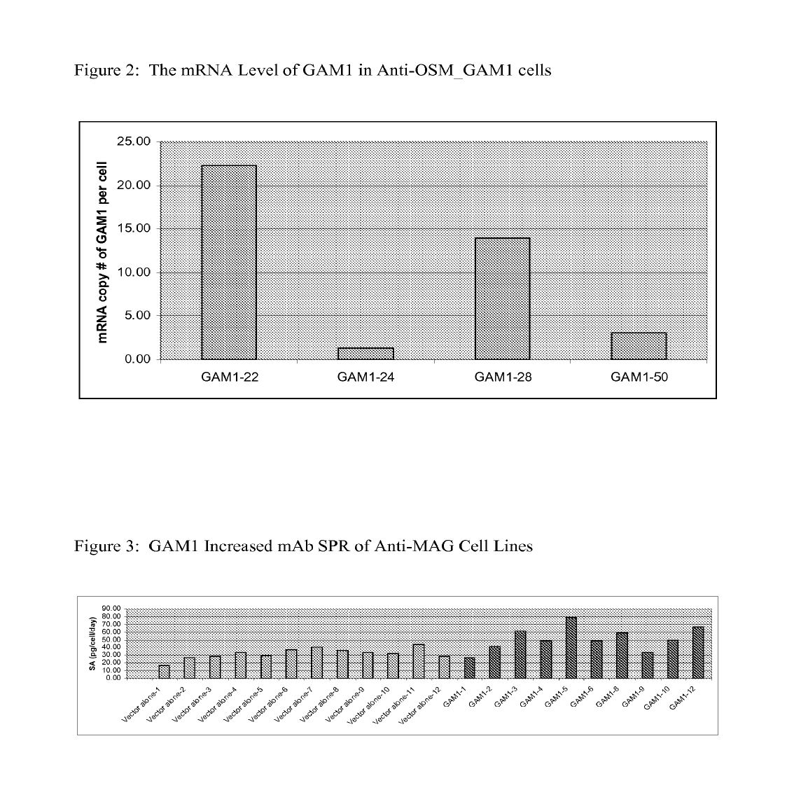

The expression level of GAM1 mRNA was measured by qRT-PCR as shown in FIG. 2. The copy numbers per cell varied among the four GAM1 clones designated as GAM1-22, 24, 28 and 50. Intermediate expression of GAM1 (3-10 copy/cell) had long term stable effects in the cells.

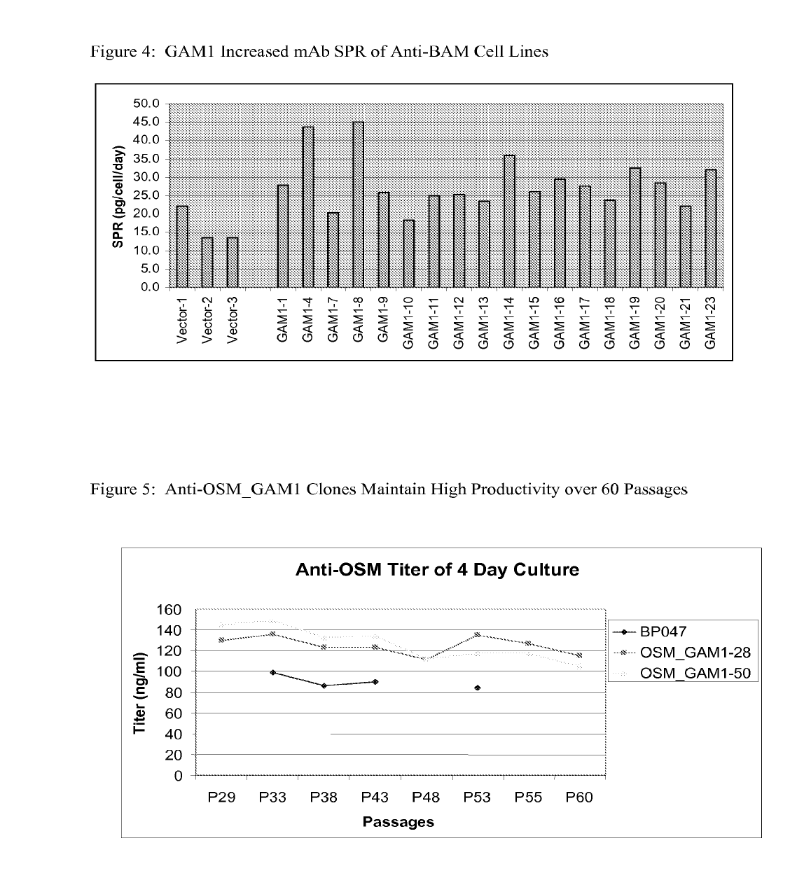

In addition, the effect of GAM1 on specific productivity of an anti-MAG antibody was also tested. A CHO cell line was previously made and produced anti-MAG monoclonal antibody at a relatively high level (SPR=32 pg/cell/day). The heavy chain and light chain of this antibody were both regulated by RSV_LTR promoters. Sequential stable transfection with GAM1 increased the SPR of anti-MAG in these cells as shown in FIG. 3. The mean SPR for anti-MAG antibody in these cells sequential stably transfected with GAM1 was 51.58 pg/cell/day, while the mean SPR for anti-MAG produced in these cells sequential transfected with vector alone was 32.2 pg/cell/day, Thus, the mean SPR for anti-MAG from these cells sequential stably transfected with GAM1 was statistically significantly higher than SPR from cells sequential transfected with vector only. Similar results were obtained for the cell lines which stably express anti-BAM monoclonal antibody as shown in FIG. 4.

### Example 2

### Increased Cell Growth and Viability

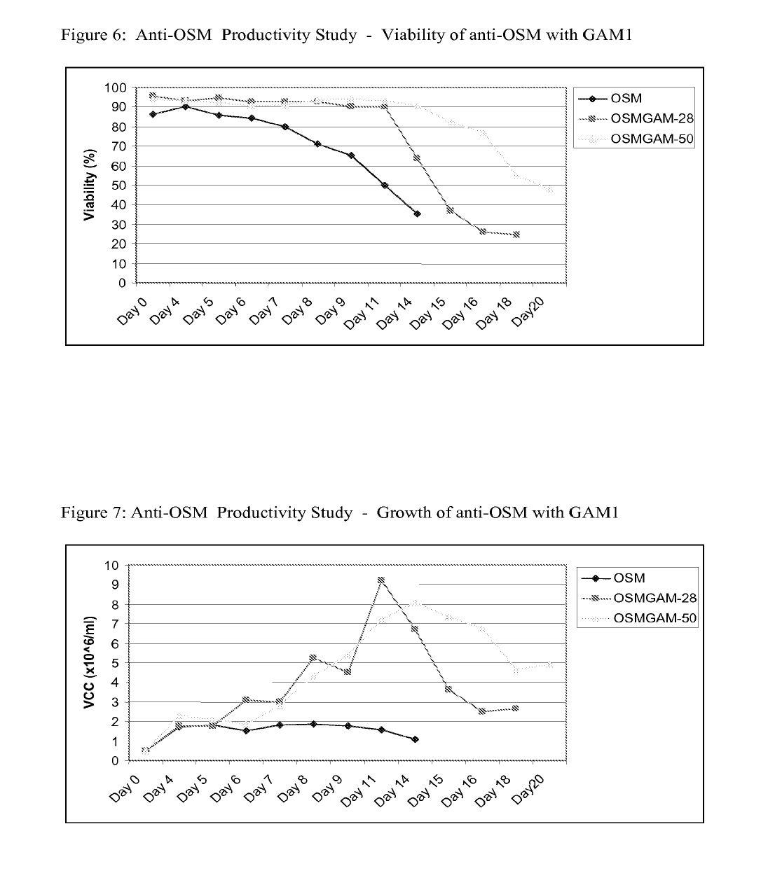

The accumulated titer of mAb production from the anti-OSM GAM1 cells of Example 1 was measured on the 4 day culture. The cells were seeded at 0.5×10⁶ cells/ml at 150 mL in a 250 mL-shaking flask. One day 4, the cells were counted and reseeded to 0.5×10⁶ cells/ml. The supernatants of each 4 day culture were quantified by ELISA. The cell lines with the expression of GAM1 maintained high productivity for over 50 passages as shown in FIG. 6.

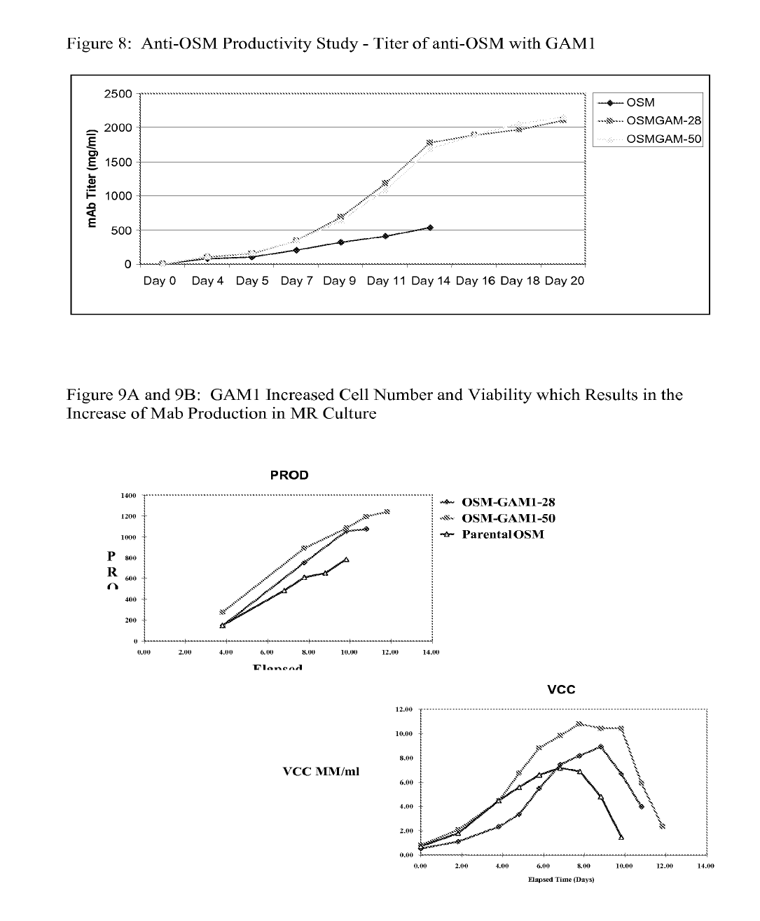

The anti-OSM GAM1 cell lines were passaged for at least 10 generations and then tested in 20-day production study in shaking flasks. The viable cell counts, percentage of viability and mAB titer were measured every 2-3 days. In comparison with the non-GAM1 cell line (OSM), GAM1 expressed cells showed a viable cell count (VCC) of greater than 8 million viable cells/mL and greater than 80% viability after day 11. In addition to the increase of specific productivity shown in Example 1, the increase of viable cell counts and delayed cell death also contribute to the increase of total antibody productivity (volumetric yield) as shown in FIGS. 6-8. The same study was repeated in mini-bio-reactors as shown in FIG. 9.

### Example 3

### Generate High Titer Mab Production Lines in a Rapid Way by Simultaneous Co-Transfection of GAM1 with Antibody Genes into CHO_D44 Cells

In this experiment, anti-beta-amyloid (BAM) heavy chain and light chain plasmids were co-transfected into CHO_DG44 and the antibody producing cells were selected by G418 in 96 well plates. In parallel, heavy chain and light chain plasmids were simultaneously co-transfected together with pcDNA_GAM1 plasmid. The cells carrying all 3 plasmids were selected by G418 and Zeo.

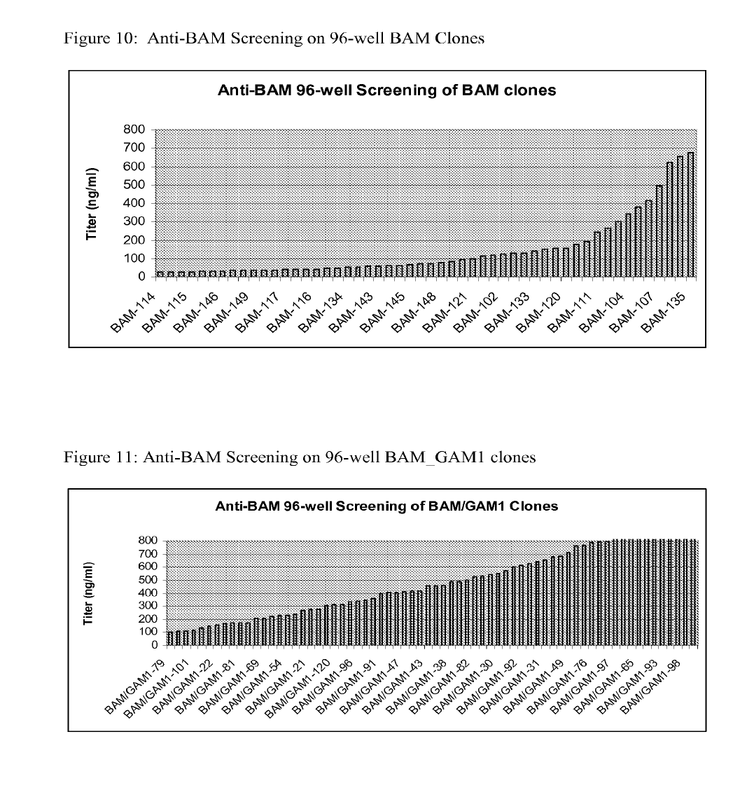

After 20 days, the plates with simultaneous GAM1 transfection had fewer colonies due to the double selection. However, among the GAM1+clones, almost all clones express mAb and about half of the clones had a titer as high as 100 ng/mL. In contrast, only ˜10% clones without simultaneous GAM1 co-expression demonstrated express the minimal amount of anti-BAM titre of 25 ng/mL. The majority of GAM130 clones grew up quickly and had a high SPRs ranging from 1.2 to 23 pg/cell/day, which is about 10 fold increase in specific productivity. Accordingly, simultaneous co-transfection of GAM1 can be used as a rapid turn-over method for mAb production. A summary of clones, Mab producing clones, and clone growth with BAM and BAM+GAM1 simultaneous cotransfection are summarized in Table 1 as well as in FIGS. 10-12.

TABLE 1

| | | | Simultaneous BAM | |
|---|---|---|---|---|
| | BAM alone Transfection | | &GAM1 Transfection | |
| Plates | 36 | | 52 | |
| G418 clones | 654 | | 269 | |
| Mab producing clones | 71 | (>25 ng/ml) | 102 | (>100 ng/ml) |
| Clones growing | 49 | (0.3-2.8 pg/c/d) | 86 | (1.2-23 pg/c/d) |

### Example 4

### Safety Assessment of GAM1

GAM1 was first identified as a novel anti-apoptotic protein of avian adenovirus CELO (Chiocca, et al., J. Virology, 71:3168-3177). Although it was originally described as an E1A like protein based on its anti-apoptotic activity, it shares no sequence homology with human adenovirus E1A. GAM1 oncogenicity was tested using NIH3T3 foci formation assay.

A retroviral-based GAM1 expression plasmid was constructed which contains the same GAM1 cDNA used in CHO cell under LTR promoter. NIH3T3 cells were infected with retroviral vector (pMSCV) or recombinant GAM1 retrovirus and selected in media containing puromycin for 48 h. The puromycin-resistant cells were suspended in DMEM containing 10% FBS and 0.4% SeaPlaque low-melting temperature agarose and plated out in 6-well plates. As positive control, retrovirus expressing Ras or its vector (pBabe) was used. Cells with no infection were used as negative control. The expression of GAM1 was confirmed both by RT-PCR and Western blot. The anchorage-independent cell growth is one hallmark of cellular transformation. The recombinant H-Ras V12 (Ras) retrovirus resulted in significant cell growth on the soft agar. However, surprisingly expression of GAM1 failed to promote cell growth. Therefore, GAM1 has no oncogenicity activity and accordingly has an enhanced safety profile compared to mammalian adenovirus E1 genes.

1. A method of increasing a first specific productivity of a recombinant antibody or fragment thereof in a first cell, comprising co-expressing said antibody or fragment thereof with adenovirus GAM1 or a fragment and/or variant thereof, wherein said first specific productivity is greater for said antibody or fragment thereof compared with a second specific productivity of the same antibody or fragment thereof expressed in a second cell, wherein said adenovirus GAM1 or fragment and/or variant thereof is not expressed.

2. (canceled)

3. The method of claim 1, wherein said antibody is humanized or is fully human.

4. (canceled)

5. (canceled)

6. The method of claim 1, wherein said antibody is a domain antibody.

7. The method of claim 1, wherein said first cell is a mammalian cell.

8. (canceled)

9. The method of claim 7, wherein said first cell is a CHO cell.

10. The method of claim 1, wherein said first cell comprises an artificial chromosome.

11. The method of claim 1, wherein said first cell comprises episome DNA.

12. The method of claim 1, wherein said first and second cell are the same.

13. (canceled)

14. The method of claim 1, wherein adenovirus GAM1 is wild type.

15. The method of claim 14, wherein said adenovirus GAM1 is avian.

16. The method of claim 1, wherein said first cell is stably transfected with DNA encoding said antibody or antibody fragment and DNA encoding said adenovirus GAM1 or fragment and/or variant thereof.

17. The method of claim 1, wherein said first cell is stably transfected with DNA encoding said antibody or antibody fragment and DNA encoding said adenovirus GAM1 or fragment and/or variant thereof is expressed from a vector in said first cell.

18. The method of claim 1, wherein said adenovirus GAM1 or fragment and/or variant thereof is present in more than one copy.

19. The method of claim 1, wherein said adenovirus GAM1 is a variant of wild type adenovirus GAM1.

20. The method of claim 19, wherein said variant of wild type adenovirus GAM1 increases the specific productivity of said monoclonal antibody or fragment thereof more than wild type adenovirus GAM1.

21. The method of claim 1, wherein said first cell is sequentially or simultaneously transfected with DNA encoding said antibody or antibody fragment and DNA encoding said adenovirus GAM1 or fragment and/or variant thereof.

22. (canceled)

23. A method of increasing integral viable cells of a first cell culture comprising co-expressing an antibody or fragment thereof with adenovirus GAM1 or a fragment and/or variant thereof in at least one cell of said first cell culture wherein said integral viable cells of said first cell culture is greater compared with the integral viable cells of a second cell culture, wherein said adenovirus GAM1 or fragment and/or variant thereof is not expressed.

24-42. (canceled)

43. A method of creating an expression stable, viable cell line comprising transfecting at least one mammalian cell with DNA encoding adenovirus GAM1 or a variant and/or fragment thereof wherein said expression stable, viable cell line comprises said at least one mammalian cell.

44. The method of claim 43, wherein said cell line remains viable and expression stable over at least about 60 passages.

45. The method of claim 44, wherein at least 1 passage occurs every 3 to 4 days.

46. The method of claim 43, wherein said cell line remains viable and expression stable for at least 210 days.

47. (canceled)

48. The method of claim 43, wherein said at least one mammalian cell is sequentially transfected with DNA encoding an antibody or fragment thereof.

49. The method of claim 43, wherein at least one mammalian cell is simultaneously or sequentially co-transfected with DNA encoding said adenovirus GAM1 or a fragment or variant thereof and DNA encoding an antibody or fragment thereof.

50-52. (canceled)

53. An expression stable, viable cell line comprising DNA encoding adenovirus GAM1 or a fragment and/or a variant thereof and capable of producing a monoclonal antibody or fragment and/or variant thereof.
